(19) United States
(12) Patent Application Publication
(10) Pub. No.: US 2026/0092309 A1
(43) Pub. Date: Apr. 2, 2026

(54) ULTRA SENSITIVE PROBES FOR DETECTING BIOMARKERS

(71) Applicant: Celnovte Biotechnology, Inc., Rockville, MD (US)

(72) Inventors: Chang Liu, Rockville, MD (US); Rajan Giri, Rockville, MD (US); Guixi Li, Rockville, MD (US); James Zhou, Rockville, MD (US); Yijun Su, Rockville, MD (US)

(21) Appl. No.: 19/236,976

(22) Filed: Jun. 12, 2025

Related U.S. Application Data
(60) Provisional application No. 63/700,794, filed on Sep. 30, 2024.

(51) Int. Cl. C12Q 1/682; C12Q 1/6804; C12Q 1/6841; C12Q 1/70

(52) U.S. Cl. CPC C12Q 1/682; C12Q 1/6841; C12Q 1/708; C12Q 1/6804

(57) ABSTRACT

The present disclosure provides compositions with ultra sensitivity for detecting a biomarker and the method of use thereof. In one embodiment, the composition comprises (1) a capture probe capable of binding to a biomarker and comprising a nucleic acid having a tail polynucleotide sequence; (2) a poly-oligonucleotide conjugate comprising a carrier conjugated with a plurality of oligonucleotides, wherein each oligonucleotide comprises a first region comprising multiple repeating polynucleotide sequences, wherein at least one of the plurality of oligonucleotides comprises a second region capable of directly or indirectly associating with the tail polynucleotide sequence; and (3) a plurality of label probes, wherein each label probe capable of directly or indirectly associating with the repeating polynucleotide sequence, wherein the capture probe, the poly-oligonucleotide conjugate and the plurality of label probes form a complex.

### CROSS-REFERENCE TO RELATED APPLICATIONS

This application claims priority to U.S. provisional application 63/700,794, filed Sep. 30, 2024, the disclosure of which is incorporated herein by reference.

### SEQUENCE LISTING

The sequence listing that is contained in the file named “089189-8001US01_SEQ”, which is 28,339 bytes and was created on Jun. 3, 2025, is filed herewith by electronic submission and is incorporated by reference herein.

### FIELD OF THE INVENTION

The present invention generally relates to cellular biology and assays. More particularly, the invention relates to probes and methods for the detection of nucleic acid or protein biomarkers in a sample.

### BACKGROUND OF THE INVENTION

Hybridization-based methods for detection of nucleic acid, such as Northern blot, Southern blot and in situ hybridization, have broad application in molecular diagnostic and biomedical research. In principle, a hybridization probe is generated by conjugating a label that provides detectable signal, such as radioactivity and fluorescence, to a fragment of DNA or RNA with sequence complementary to a target sequence. The hybridization probe hybridizes to single-stranded nucleic acid (DNA or RNA) containing the target sequence due to complementarity between the probe and target. The signal from the label is detected to determine the presence or absence of the target sequence. However, the application of hybridization probe can be limited by its inability to detect DNA or RNA targets with low copy numbers due to lack of sensitivity and specificity.

Branched-DNA (bDNA) technology is a powerful and sensitive method used for the detection and quantification of nucleic acids, including mRNA and DNA biomarkers, in various types of samples, such as Formalin-Fixed, Paraffin-Embedded (FFPE) tissue samples. In this method, as illustrated in FIG. 1, target probes (also called capture probe, shown in FIG. 1 as Z probes) are applied to hybridize with the target mRNA or DNA sequences. The target probe consists of a region that is complementary to the target sequence, as well as a region that is complementary to a pre-amplifier probe, which is applied to hybridize with the capture probe after the target probe step. The pre-amplifier probe consists of a region to be complementary to the target probe, as well as multiple binding sites for the amplifier probe. These binding sites are repeating sequences separated by spacer sequences, wherein each binding site can hybridize with one amplifier probe. After the pre-amplifier step, the amplifier probe is applied to hybridize with the pre-amplifier probe. The amplifier probe consists of a region to be complementary to the pre-amplifier probe, as well as multiple binding sites for the labeled probe. Just as the pre-amplifier probe, in the amplifier probe the binding sites are repeating sequences separated by spacer sequences, each binding site can hybridize with one labeled probe. As a result, multiple labeled probes can bind to the target sequences, generating amplified detectable signals.

However, the sensitivity of the branched-DNA methods is limited by the sequence length limitation of the synthesized oligonucleotide probe. For standardized solid-phase synthesis methods, the efficiency of chemical synthesis of oligonucleotides decreases as the length of the sequence increases. It is usually difficult to synthesize oligonucleotide probes of more than 200 nucleotides. This limits the number of repeating sequences on the components of the branched-DNA technology as each repeating sequence is usually 15-30 nucleotides in length (e.g. pre-amplifier probe, amplifier probe), thus limiting the sensitivity of this technology. Although there are enzymatic methods to extend oligonucleotide probes to be longer, these long single-stranded DNA probes tend to hybridize more slowly due to their bulky size and unfavored kinetics, which can result in unsatisfactory sensitivity in the assay. Therefore, there is continuing need to develop probes with ultra sensitivity to detect nucleic acids.

### BRIEF SUMMARY OF THE INVENTION

In one aspect, the present disclosure provides a method of detecting a biomarker in a sample. In some embodiments, the method comprises the steps of: (a) applying a capture probe to the sample, wherein the capture probe specifically binds to the biomarker and comprises a nucleic acid having a tail polynucleotide sequence; (b) applying a poly-oligonucleotide conjugate to the sample, wherein the poly-oligonucleotide conjugate comprises a carrier conjugated with a plurality of oligonucleotides, wherein each oligonucleotide comprises a first region comprising multiple repeating polynucleotide sequences, wherein at least one of the plurality of oligonucleotides comprises a second region capable of directly or indirectly associating with the tail polynucleotide sequence, thereby associating the poly-oligonucleotide conjugate with the capture probe bound to the target biomarker in step (a); (c) applying a plurality of label probes to the sample, wherein each label probe capable of directly or indirectly associating with the repeating polynucleotide sequence, thereby associating the plurality of label probes with the poly-oligonucleotide conjugate bound to the capture probe in step (b); and (d) detecting the label probes associated with the poly-oligonucleotide conjugate in step (c).

In some embodiments, the biomarker is a nucleic acid. In some embodiments, the nucleic acid is selected from the group consisting of a genomic DNA, a cDNA, a mRNA, a rRNA, a miRNA, a Lnc RNA and a siRNA. In certain embodiments, the nucleic acid is a single-stranded DNA or RNA.

In some embodiments, the biomarker is a protein. In some embodiments, the capture probe comprises an antibody against the protein.

The tail polynucleotide sequence consists of 10-40 nucleotides (e.g. 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 nucleotides).

In some embodiments, the carrier is a polymer, a nanoparticle, a bead, a liposome or a micelle. In some embodiments, the polymer is selected from the group consisting of polysaccharides such as dextrans, derivatized dextrans, cyclodextrins, pullulans, schizophyllan, scleroglucan, xanthan, chitins, chitosans, carrageenans, alginates, agarose, ficoll, derivatized ficoll, derivatized cellulosics, derivatized starch; vinyl polymers such as poly(acrylic acid), poly(acryl amides), poly(acrylic esters), poly(2-hydroxyethyl methacrylate), poly(methyl methacrylate), poly(maleic acid), poly(maleic anhydride), poly(acrylamide), poly(ethyl-co-vinyl acetate), poly(methacrylic acid), poly(vinyl alcohol), poly(vinyl alcohol-co-vinyl chloroacetate), aminated poly(vinyl alcohol), and co-block polymers thereof; polyethylene glycol, polypropylene glycol and their derivatives; block co-polymers such as poly(ethylene oxide-co-propylene oxide); hyperbranched polymers such as PAMAM dendrimers; Poly amino acids such as poly-lysine, polyglutamic acid, polyurethanes and poly(ethylene imines); Proteins such as albumins and immunoglobulins; or mixed polymers comprised of one or more polymers listed above.

In some embodiments, thioether linkage is utilized to conjugate a carrier to nucleic acid. In some embodiments, the linkage used can be amide, thiourea, reductive amination via imine, hydrazone, oxime, glyoxylic-oxime, disulfide, thiazolidine, native ligation, Diels-Alder cycloaddition, Huisgen 1,3-dipolar cycloaddition and Staudinger ligation.

In some embodiments, the repeating polynucleotide sequence consists of 10-40 nucleotides (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 nucleotides).

In some embodiments, at least one of the plurality of oligonucleotides comprises a second region capable of directly or indirectly associating with the tail polynucleotide sequence, thereby associating the poly-oligonucleotide conjugate with the capture probe bound to the target biomarker. In some embodiments, the second region is complementary to the tail polynucleotide sequence, thus associating with the tail polynucleotide sequence directly. In some embodiments, the second region is capable of associating with the tail polynucleotide sequence via at least one bridge probe. In some embodiments, the bridge probe comprises sequences capable of hybridizing to the second region and the tail polynucleotide sequence, respectively. In some embodiments, the second region is capable of associating with the tail polynucleotide sequence via multiple bridge probes, wherein the multiple bridge probes hybridize with each other, at least one bridge probe hybridizes with the second region and at least another bridge probe hybridizes with the tail polynucleotide sequence.

In some embodiments, the label probe has a sequence capable of hybridizing to the repeating polynucleotide sequence. In some embodiments, the label probe is capable of associating with the repeating polynucleotide sequence via at least one bridge probe. In some embodiments, the bridge probe has regions capable of hybridizing to the repeating polynucleotide sequence and the label probe, respectively.

In some embodiments, the label probe comprises a detectable label. In some embodiments, the detectable label is a fluorophore, a horse radish peroxidase or an alkaline phosphatase.

In some embodiments, the sample is body fluid (e.g., sera, plasma, saliva, urine), cell or tissue.

In certain embodiments, the probe (e.g., the capture probe, label probe) and the oligonucleotide described herein can comprise one or more nucleotide analogs (e.g., altered backbone, sugar, or nucleobase). In certain embodiments, the nucleotide analog is selected from the group consisting of 5-bromouracil, a peptide nucleic acid nucleotide, a xeno nucleic acid nucleotide, a morpholino, a locked nucleic acid nucleotide, a glycol nucleic acid nucleotide, a threose nucleic acid nucleotide, a dideoxynucleotide, a cordycepin, a 7-deaza-GTP, a fluorophore (e.g. rhodamine or flurescein linked to the sugar), a thiol containing nucleotide, a biotin linked nucleotide, a fluorescent base analog, a methyl-7-guanosine, a methylated nucleotide, an inosine, thiouridine, a pseudourdine, a dihydrouridine, a queuosine, and a wyosine. In certain embodiments, the nucleotide analog is a locked nucleic acid nucleotide.

In another aspect, the present disclosure provides a composition of detecting a biomarker in a sample. In some embodiments, the composition comprises: (a) a capture probe capable of binding to a biomarker and comprising a nucleic acid having a tail polynucleotide sequence; (b) a poly-oligonucleotide conjugate comprising a carrier conjugated with a plurality of oligonucleotides, wherein each oligonucleotide comprises a first region comprising multiple repeating polynucleotide sequences, wherein at least one of the plurality of oligonucleotides comprises a second region capable of directly or indirectly associating with the tail polynucleotide sequence; and (c) a plurality of label probes, wherein each label probe capable of directly or indirectly associating with the repeating polynucleotide sequence, wherein the capture probe, the poly-oligonucleotide conjugate and the plurality of label probes form a complex.

In another aspect, the present disclosure provides a kit of detecting a biomarker in a sample. In some embodiments, the kit comprises: (a) a capture probe capable of binding to a biomarker and comprising a nucleic acid having a tail polynucleotide sequence; (b) a poly-oligonucleotide conjugate comprising a carrier conjugated with a plurality of oligonucleotides, wherein each oligonucleotide comprises a first region comprising multiple repeating polynucleotide sequences, wherein at least one of the plurality of oligonucleotides comprises a second region capable of directly or indirectly associating with the tail polynucleotide sequence; and (c) a plurality of label probes, wherein each label probe capable of directly or indirectly associating with the repeating polynucleotide sequence, wherein the capture probe, the poly-oligonucleotide conjugate and the plurality of label probes are capable of forming a complex when mixed.

### BRIEF DESCRIPTION OF THE FIGURES

The following drawings form part of the present specification and are included to further demonstrate certain aspects of the present disclosure. The disclosure may be better understood by reference to one or more of these drawings in combination with the detailed description of specific embodiments presented herein.

FIG. 1 shows a schematic of a branched in situ hybridization method.

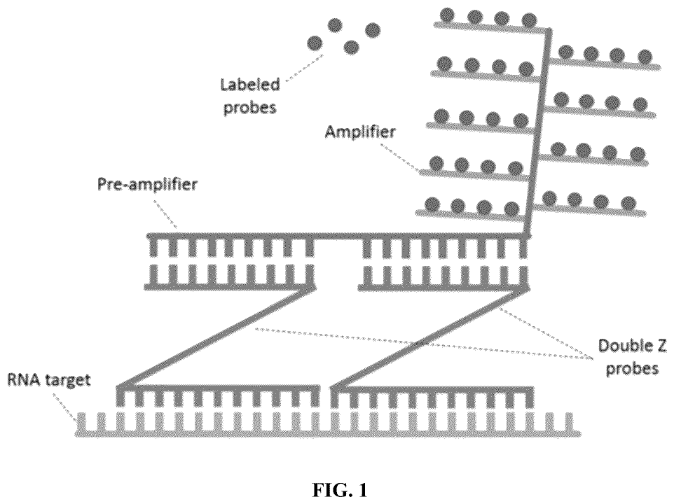

FIG. 2 shows an exemplary embodiment of ultra-sensitive probe composition described herein.

FIG. 2A shows an exemplary embodiment of the poly-oligonucleotide conjugate described herein.

FIG. 3 shows the flowchart of an exemplary method of using the ultra-sensitive probe composition described herein to detect a biomarker in a sample.

FIG. 4 show the assembly and application of an ultra-sensitive probe composition according to an embodiment of the invention. a. the framework of the control sample. b the framework of the test sample containing poly-oligonucleotide conjugate.

FIG. 5 shows the results of validation of poly-nucleotide conjugate staining. a, b staining result of positive control on two different low-risk HPV tissues. c, d staining result of the tested poly-nucleotide conjugate on the same low-risk HPV tissues.

FIG. 6 shows the results of validation of poly-nucleotide conjugate staining, wherein the label probe indirectly associated with the poly-oligonucleotide conjugate through the amplifier probe. a, b staining result of positive control on two different low-risk HPV tissues. c, d staining result of the tested poly-nucleotide conjugate on the same low-risk HPV tissues.

FIG. 7 shows the results of validation of poly-nucleotide conjugate staining, wherein the capture probe is an oligonucleotide conjugated antibody, and the label probe is a fluorescence labelled oligonucleotide. a. the bar graph displays the comparison of the signal to background ratio of the control slide and the test slide. b. staining results of positive control on human tonsil tissue, wherein the contrast of image was independently adjusted to show the localization of label due to its significant brighter signal strength. c. staining results of the tested poly-nucleotide conjugate on the same human tonsil tissue.

### DETAILED DESCRIPTION OF THE INVENTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

### Definitions

The following definitions are provided to assist the reader. Unless otherwise defined, all terms of art, notations and other scientific or medical terms or terminology used herein are intended to have the meanings commonly understood by those of skill in the chemical and medical arts. In some cases, terms with commonly understood meanings are defined herein for clarity and/or for ready reference, and the inclusion of such definitions herein should not necessarily be construed to represent a substantial difference over the definition of the term as generally understood in the art.

As used herein, the singular forms “a,” “an” and “the” include plural references unless the content clearly dictates otherwise. The term “based on” is not exclusive and allowed for being based on additional factors not described unless the context clearly dictates otherwise. The meaning of “in . . . ” includes “within . . . ” and “on . . . ”. The term “or” is an inclusive “or” operator and is equivalent to the term “and/or”, unless the context clearly dictates otherwise.

The term “comprises” and grammatical equivalents thereof are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also one or more other components.

As used herein, the terms “antibody” and “immunoglobulin” may generally refer to proteins that can recognize and bind to a specific antigen. An antibody or immunoglobulin may refer to an antibody isotype, fragments of antibodies including, but not limited to, Fab, Fv, scFv, and Fd fragments, chimeric antibodies, humanized antibodies, single-chain antibodies, and fusion proteins including an antigen-binding portion of an antibody and a non-antibody protein. The antibodies may be detectably labeled, e.g., with a fluorophore, radioisotope, enzyme (e.g., a peroxidase) which generates a detectable product, fluorescent protein, nucleic acid barcode sequence, and the like. The antibodies may be further conjugated to other moieties, such as members of specific binding pairs, e.g., biotin (member of biotin-avidin specific binding pair), and the like. Also encompassed by the terms are Fab′, Fv, F(ab′)2, and other antibody fragments that retain specific binding to antigen. Antibodies may exist in a variety of other forms including, for example, Fv, Fab, and (Fab)2, as well as bi-functional (i.e., bi-specific) hybrid antibodies (e.g., Lanzavecchia et al., Eur. J. Immunol. 17, 105 (1987)) and in single chains (e.g., Huston et al., Proc. Natl. Acad. Sci. U.S.A., 85, 5879-5883 (1988) and Bird et al., Science, 242, 423-426 (1988), which are incorporated herein by reference). (See, generally, Hood et al., Immunology, Benjamin, N.Y., 2nd ed. (1984), and Hunkapiller and Hood, Nature, 323, 15-16 (1986), which are herein incorporated by reference).

As used herein, “associate” or “associating” means physically direct or indirect attachment. For example, the label probe can hybridize to the poly-oligonucleotide conjugate, which hybridizes to the capture probe, which hybridizes the target nucleic acid, thereby the label probe is associated with the target nucleic acid.

The term “at least” followed by a number is used herein to denote the start of a range beginning with that number (which may be a range having an upper limit or no upper limit, depending on the variable being defined). For example, “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. When, in this specification, a range is given as “(a first number) to (a second number)” or “(a first number)-(a second number),” this means a range whose lower limit is the first number and whose upper limit is the second number. For example, 4 to 20 nucleotides means a range whose lower limit is 4 nucleotides, and whose upper limit is 20 nucleotides.

The term “complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%>, 70%>, 80%>, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions. In could be understood that the term “complementary” as used in the present disclosure include both perfectly complementary and substantially complementary unless the context indicates otherwise.

The term “conjugate” as used herein refers a complex or composition matters comprising two or more molecules, components, or parts that are jointed, linked, associated, coupled, connected or related together. The joining, linking, associating, coupling, connecting, or relating can be covalent or non-covalent.

The term “hybridizing” refers to the binding, duplexing, or hybridizing of a nucleic acid molecule preferentially to a particular nucleotide sequence under stringent conditions. The term “stringent conditions” refers to conditions under which a probe will hybridize preferentially to its target subsequence, and to a lesser extent to, or not at all to, other sequences in a mixed population (e.g., a cell lysate or DNA preparation from a tissue biopsy). A “stringent hybridization” and “stringent hybridization wash conditions” in the context of nucleic acid hybridization (e.g., as in array, microarray, Southern or northern hybridizations) are sequence dependent, and are different under different environmental parameters. An extensive guide to the hybridization of nucleic acids is found in, e.g., Tijssen Laboratory Techniques in Biochemistry and Molecular Biology—Hybridization with Nucleic Acid Probes part I, Ch. 2, “Overview of principles of hybridization and the strategy of nucleic acid probe assays,” (1993) Elsevier, N.Y. Generally, highly stringent hybridization and wash conditions are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Very stringent conditions are selected to be equal to the Tm for a particular probe. An example of stringent hybridization conditions for hybridization of complementary nucleic acids which have more than 100 complementary residues on an array or on a filter in a Southern or northern blot is 42° C. using standard hybridization solutions (see, e.g., Sambrook and Russell Molecular Cloning: A Laboratory Manual (3rd ed.) Vol. 1-3 (2001) Cold Spring Harbor Laboratory, Cold Spring Harbor Press, NY). An example of highly stringent wash conditions is 0.15 M NaCl at 72° C. for about 15 minutes. An example of stringent wash conditions is a 0.2×SSC wash at 65° C. for 15 minutes. Often, a high stringency wash is preceded by a low stringency wash to remove background probe signal. An example medium stringency wash for a duplex of, e.g., more than 100 nucleotides, is 1×SSC at 45° C. for 15 minutes. An example of a low stringency wash for a duplex of, e.g., more than 100 nucleotides, is 4×SSC to 6×SSC at 40° C. for 15 minutes.

As used herein, the term “nucleic acid” (interchangeable with the term “polynucleotide”) encompasses any physical string of monomer units that can be corresponded to any physical string of monomer units that can be corresponded to a string of nucleotides, including a polymer of nucleotides (e.g., a typical DNA or RNA polymer), peptide nucleic acids (PNAs), modified oligonucleotides (e.g., oligonucleotides comprising nucleotides that are not conventional to biological RNA or DNA, such as 2′-O-methylatded oligonucleotides), and the like. The nucleic acid can be both single-stranded and double-stranded. The nucleotides of the polynucleotide can be deoxyribonucleotides, ribonucleotides or nucleotide analogs, can be natural and unnatural, and can be unsubstituted, unmodified, substituted or modified. The nucleotides can be linked by phosphodiester bonds, or by phosphorothioate linkages, methylphosphonate linkages, boranophosphate linkages, or the like. The polynucleotide can additionally comprise non-nucleotide elements such as labels, quenchers, blocking groups, or the like. The polynucleotide can be, e.g., single-stranded or double-stranded.

As used herein, a “nucleotide analog” refers to a nucleotide (deoxyribonucleotide or ribonucleotide) comprising one or more modifications (e.g. altered backbone, sugar, or nucleobase). Some non-limiting examples of nucleotide analogs include: 5-bromouracil, peptide nucleic acid nucleotides, xeno nucleic acid nucleotides, morpholinos, locked nucleic acid nucleotides, glycol nucleic acid nucleotides, threose nucleic acid nucleotides, dideoxynucleotides, cordycepin, 7-deaza-GTP, florophores (e.g., rhodamine or flurescein linked to the sugar), thiol containing nucleotides, biotin linked nucleotides, fluorescent base analogs, CpG islands, methyl-7-guanosines, methylated nucleotides, inosines, thiouridines, pseudourdines, dihydrouridines, queuosines, and wyosines.

Xeno nucleic acid (XNA) refers to a group of synthetic polymers similar to DNA and RNA that differ in the sugar backbone. Examples of XNA include without limitation 1,5-anhydrohexitol nucleic acid (HNA), cyclohexene nucleic acid (CeNA), Threose nucleic acid (TNA), glycol nucleic acid (GNA), locked nucleic acid (LNA), peptide nucleic acid (PNA).

Peptide nucleic acid (PNA) is an artificial synthesized polymer similar to DNA or RNA. While DNA and RNA have a deoxyribose and ribose sugar backbone, respectively, PNA's backbone is composed of repeating N-(2′ aminoethyl)-glycine units linked by peptide bonds. The various purine and pyrimidine bases are linked to the backbone by a methylene bridge (—CH₂—) and a carbonyl group (—(C═O)—). Because the backbone of PNA contains no charged phosphate groups, the binding between PNA/DNA strands is stronger than between DNA/DNA strands due to the lack of electrostatic repulsion, resulting in increased melting temperature.

A locked nucleic acid is a modified RNA nucleotide whose ribose moiety is modified with an extra bridge connecting the 2′ oxygen and 4′ carbon. The bridge “locks” the ribose in 3′-endo conformation, which enhances base stacking and backbone pre-organization of the locked nucleic acid, thus significantly increases its hybridization properties (melting temperature).

Threose nucleic acid (TNA) has a backbone structure composed of repeating threose sugars linked together by phosphodiester bonds. TNA can self-assemble by Wastson-Crick base pairing into duplex structure and can form base pairs complementary to strands of DNA and RNA.

Glycol nucleic acid (GNA) has a backbone composed of repeating glycol units linked by phosphodiester bonds. GNA shows a stronger Watson-Crick base pairing than DNA and RNA and requires a high temperature to melt a duplex GNA or GNA/DNA, GNA/RNA.

A “nucleic acid target” or “target nucleic acid” means a nucleic acid, or optionally a region thereof, that is to be detected. The target nucleic acid can have a nucleic acid sequence existing in the nature or any sequence designed and generated by human. For example, the nucleic acid sequence can be a genomic sequence of a prokaryotic or eukaryotic species. A prokaryotic species includes, for example, bacteria. A eukaryotic species includes, for example, a fungus, a plant, an animal, e.g., a mammal. In particular, the sequence of a target nucleic acid of interest can be found in public available databases, for example, the database of National Center for Biotechnology Information. The target nucleic acid can be single-stranded or double stranded. In certain embodiments, the target nucleic acid is a single stranded nucleotide polymer. In certain embodiments, the target nucleic acid is a single-stranded DNA or RNA (e.g., mRNA, siRNA, LncRNA). In certain embodiments, the target nucleic acid has 15 or more nucleotides, e.g., 20, 25, 30, 35, 40, 50, 60, 70, 80, 90, 100, 200, 300, 400, 500, 600, 700, 800, 900, 1000, 2000, 3000, 4000, 5000 or more nucleotides.

As used herein, a “nucleotide sequence” or “polynucleotide sequence” is a polymer of nucleotides (an oligonucleotide, a DNA, a nucleic acid, etc.) or a character string representing a nucleotide polymer, depending on context. From any specified nucleotide sequence, either the given nucleic acid or the complementary nucleic acid sequence can be determined.

A “label” as used herein is a moiety that facilitates detection of a molecule, typically by directly or indirectly providing a detectable signal. Common labels in the context of the present invention include fluorescent, luminescent, light-scattering, and/or colorimetric labels. Suitable labels may be selected from fluorescent labels such as 5-(and 6)-carboxyfluorescein, 5- or 6-carboxyfluorescein, 6-(fluorescein)-5-(and 6)-carboxamido hexanoic acid, fluorescein isothiocyanate (FITC), rhodamine, tetramethylrhodamine, and dyes such as Cy2, Cy3, and Cy5, optionally substituted coumarin including AMCA, PerCP, phycobiliproteins including R-phycoerythrin (RPE) and allophycoerythrin (APC), Texas Red, Princeston Red, Green fluorescent protein (GFP) and analogues thereof, and conjugates of R-phycoerythrin or allophycoerythrin and e.g. Cy5 or Texas Red, and inorganic fluorescent labels based on semiconductor nanocrystals (like quantum dot and Qdot™ nanocrystals), and time-resolved fluorescent labels based on lanthanides like Eu3+ and Sm3+, haptens such as DNP, biotin, and digoxiginin, protein labels with strong and specific binding properties such as streptavidin, modified or engineered streptavidin, avidin, modified or engineered avidin, nanobodies, affibodies, anticalins, Snap-Tag and HaloTag systems, enzymatic labels such as horse radish peroxidase (HRP), alkaline phosphatase (AP), beta-galactosidase (GAL), glucose-6-phosphate dehydrogenase, beta-N-acetyl-glucosaminidase, ß-glucuronidase, invertase, Xanthine Oxidase, firefly luciferase and glucose oxidase (GO), luminiscence labels such as luminol, isoluminol, acridinium esters, 1,2-dioxetanes and pyridopyridazines, radioactivity labels such as incorporated isotopes of iodide, cobalt, selenium, tritium, and phosphor, and single atoms such as zinc (Zn), iron (Fe), magnesium (Mg), any of the lanthanides (Ln) including La, Ce, Pr, Nd, Pm, Sm, Eu, Gd, Tb, Dy, Ho, Er, Tm, Yb and Lu; scandium (Sc) and yttrium (Y). Suitable labels also include a moiety absent from a target sample, such as a hapten (e.g., digoxin), which can be further detected with immunoassay methods using an anti-hapten antibody.

As used herein, a “pre-amplifier probe” refers to an oligonucleotide sequence that binds to the capture probe, directly or indirectly. Pre-amplifier probe is typically a single-stranded polynucleotide that comprises a region that can associate with the tail polynucleotide sequence of the capture probe. The pre-amplifier probe serves as a bridge between capture probe and label probe and can be used as signal amplification component.

As used herein, a “label probe” refers to an entity that binds to a target molecule, directly or indirectly, and enables the target molecule to be detected, e.g., by a readout instrument. A label probe is typically a single-stranded polynucleotide that comprises one or more label that directed or indirectly provides a detectable signal. The label can be covalently linked to the polynucleotide, or the polynucleotide can be configured to bind to the label (e.g., a biotinylated polynucleotide can bind a streptavidin associated label).

The label probe can, for example, hybridize directly to a target nucleic acid, or it can hybridize to a nucleic acid (e.g., a poly-oligonucleotide conjugate) that is in turn hybridized to the target nucleic acid or to one or more other nucleic acids that are hybridized to the nucleic acid. In preferred embodiments, the label probe can comprise a nucleotide sequence that is substantially complementary (e.g., at least 90% complementary, at least 95% complementary, at least 99% complementary, 100% complementary) to a nucleotide sequence (e.g., a gap nucleotide sequence) in a poly-oligonucleotide conjugate, or the like.

As used herein, a “probe” is an entity that can be used in the detection of a target molecule. Typically, a probe in the present disclosure refers to a nucleic acid molecule, with or without modification. The probe can be both single-stranded and double-stranded nucleotide polymers. Unless indicated otherwise, the probes described in the present application is a single-stranded nucleotide polymer.

The term “sample” as used herein refers to any sample having or suspect of having the target biomarker, including sample of biological tissue or fluid origin, obtained, reached, or collected in vivo or in situ. Exemplary biological samples include but are not limited to cell lysate, a cell culture, a cell line, a tissue, an organ, a biological fluid, and the like. In certain embodiments, the sample is selected from the group consisting of sera, plasma, saliva, urine, cell and tissue.

### Composition for Detecting Biomarkers

Ultra-sensitive probes are useful in molecular diagnostic and biomedical research, especially in detecting biomarkers of low abundance in a sample. Branched in situ hybridization is a technology provides a sensitive method for detecting nucleic acids of low abundance in various types of samples. However, the sensitivity of the branched-DNA methods is limited by the sequence length of the synthesized oligonucleotide probe. Therefore, the present disclosure in one aspect provides a probe composition with ultra sensitivity for detecting biomarkers.

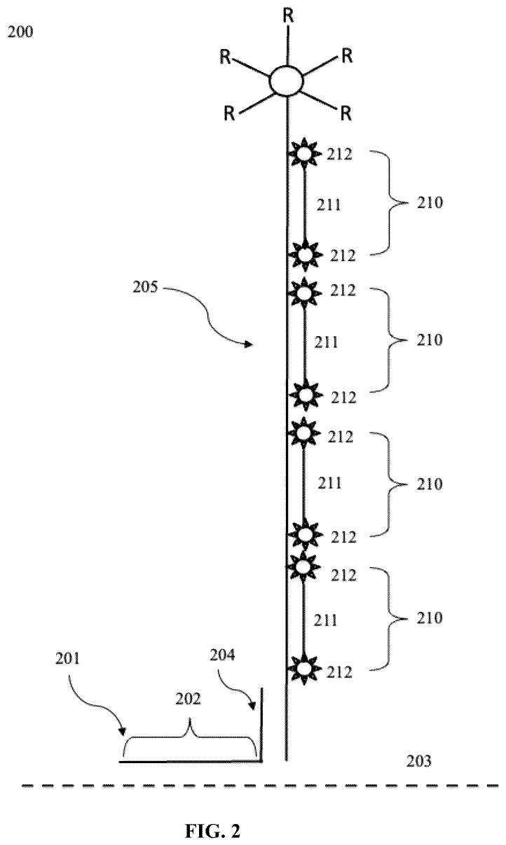

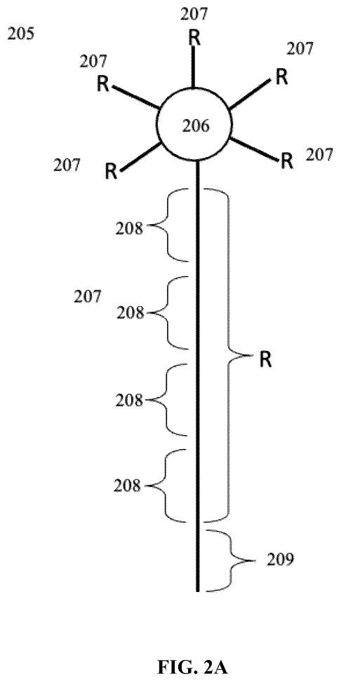

An exemplary embodiment of ultra-sensitive probe composition described herein is illustrated in FIG. 2. Referring to FIG. 2, the probe composition 200 is composed of a capture probe 201 comprising a moiety 202 capable of binding to a biomarker 203. In some embodiments, the biomarker 203 is a nucleic acid, e.g., DNA or mRNA. In such case, the moiety 202 is a polynucleotide capable of hybridizing to the biomarker 203. Typically, the moiety 202 includes a nucleotide sequence that has 10-40 nucleotides and is complementary to a sequence of the biomarker 203. In some embodiments, the biomarker 203 is a protein. In such case, the moiety 202 could be an antibody or aptamer capable of specifically binding to the biomarker 203. In some embodiments, the capture probe is a single-stranded nucleic acid (e.g. DNA or RNA). In some embodiments, the capture probe comprises a single-stranded nucleic acid coupled to an antibody or aptamer.

Now referring to FIG. 2, the capture probe 201 further comprises a tail polynucleotide sequence 204 capable of binding to a poly-oligonucleotide conjugate 205. An exemplary embodiment of the poly-oligonucleotide conjugate described herein is illustrated in FIG. 2A. Now referring to FIG. 2A, the poly-oligonucleotide conjugate 205 comprises a carrier 206 conjugated with a plurality of oligonucleotides 207, wherein each oligonucleotide 207 comprises a first region R comprising multiple repeating polynucleotide sequences 208.

A carrier 206 can be any complex, compound, particle or substrate suitable for coupling multiple oligonucleotides. In some embodiment, the carrier 206 is a polymer. In some embodiments, the carrier 206 is a polymer with a plurality of maleamide groups. Thiolated oligonucleotides can react with the maleamide group to produce the poly-oligonucleotide conjugate 205. Synthesized poly-oligonucleotide conjugate 205 can be purified with size-exclusion chromatography and analyzed by high pressure liquid chromatography to ensure purity and conjugate-consistency.

Typically, each repeating polynucleotide sequence 208 has 10-40 nucleotides (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 nucleotides) and each region R has at least one repeating polynucleotide sequences. In a poly-oligonucleotide conjugate 205, at least one of the plurality of oligonucleotides 207 comprises a second region 209 complementary to the tail polynucleotide sequence, thus allowing the poly-oligonucleotide conjugate 205 to hybridize to a capture probe 201, as illustrated in FIG. 2.

Now referring to FIG. 2, the probe composition 200 further comprises a plurality of label probes 210. Each label probe 210 has a polynucleotide sequence 211 complementary to the repeating polynucleotide sequence 208, thus allowing multiple label probes 210 to bind to a poly-oligonucleotide conjugate 205. Each label probe 210 further comprises one or more label 212 coupled to the polynucleotide 211 wherein the label 212 directly or indirectly provides a detectable signal. The label 212 can be covalently linked to the polynucleotide 211, or the polynucleotide 211 can be configured to bind to the label 212 (e.g., a biotinylated polynucleotide can bind a streptavidin associated label).

### Method of Use

In another aspect, the present disclosure provides methods of using the ultra-sensitive probe compositions in detecting biomarkers.

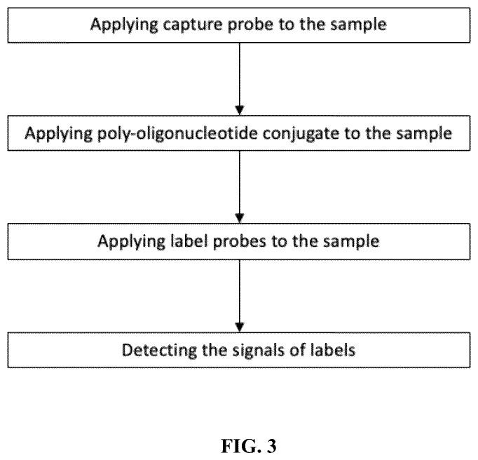

An exemplary embodiment of the method of using the ultra-sensitive probe composition described herein is illustrated in FIG. 3. Referring to FIG. 3, in the first step, the method comprises applying a capture probe to the sample, wherein the capture probe specifically binds to the biomarker, e.g., via nucleic acid hybridization when the biomarker is a nucleic acid. As described elsewhere herein, the capture probe also comprises a nucleic acid having a tail polynucleotide sequence capable of hybridizing to a poly-oligonucleotide conjugate. After the capture probe step, the method comprises applying to the sample the poly-oligonucleotide conjugate which comprises a carrier conjugated with a plurality of oligonucleotides. As described elsewhere herein, each oligonucleotide comprises a first region comprising multiple repeating polynucleotide sequences, and at least one of the plurality of oligonucleotides comprises a second region complementary to the tail polynucleotide sequence, thereby associating the poly-oligonucleotide conjugate with the capture probe bound to the target biomarker. After the poly-oligonucleotide conjugate step, the method further comprises applying a plurality of label probes to the sample, wherein each label probe has a polynucleotide sequence complementary to the repeating polynucleotide sequence, thereby associating the plurality of label probes with the poly-oligonucleotide conjugate bound to the capture probe. After the label probe step, the method further comprises detecting the label probes associated with the poly-oligonucleotide conjugate.

In could be understood that the method described herein may comprise additional steps. For example, the method may comprise removing excessive capture probes not bound to the biomarker after the capture probe step. Similarly, the method may comprise removing poly-oligonucleotide conjugate not bound to the capture probe after the poly-oligonucleotide conjugate step and removing excessive label probes not bound to the poly-oligonucleotide conjugate after the label probe step.

### Kits

Also provided herein are kits for detecting a biomarker in a sample. The kits provided herein may be useful in implementing any of the described methods or may be provided in complement to the described methods.

In one aspect, a kit of the present disclosure may comprise the reagents for forming the ultra-sensitive probe composition described herein. In some embodiments, the kit comprises a capture probe capable of binding to a biomarker, wherein the capture probe comprises a tail polynucleotide sequence. The kit further comprises a poly-oligonucleotide conjugate comprising a carrier conjugated with a plurality of oligonucleotides, wherein each oligonucleotide comprises a first region comprising multiple repeating polynucleotide sequences and at least one of the plurality of oligonucleotides comprises a second region complementary to the tail polynucleotide sequence. The kit also comprises a label probe having a polynucleotide sequence complementary to the repeating polynucleotide sequence.

The kits may further comprise buffers, reagents, binding agents, catalysts, or other chemicals or biological molecules (e.g., enzymes) necessary for conducting the methods described herein. In one embodiment, the kit may comprise reagents for detecting a hapten label (e.g., digoxin), such as an anti-hapten antibody. The kits may further comprise instructions for using the components of the kit or for implementing any of the methods and processes described herein.

While the disclosure has been particularly shown and described with reference to specific embodiments (some of which are preferred embodiments), it should be understood by those having skill in the art that various changes in form and detail may be made therein without departing from the spirit and scope of the present disclosure as disclosed herein.

### EXAMPLES

The following examples, not intended to limit the scope of present invention, will illustrate the manufacture and application of components claimed.

### Example 1

This example details the synthesis of poly-oligonucleotide conjugated using thiol-oligonucleotide and commercially available maleamide-terminated PEG carrier.

The thiol group on the nucleic acid was generated as follows. 5′-thiol modified nucleic sequence (synthesized by GenScript Biotech) was dissolved in tris-EDTA (TE) buffer (pH 8) to give 10 uM solution. The reduction was carried out in aqueous condition using dithiothreitol (DTT). 500 mM DTT solution was prepared in 1 ml TE buffer. Appropriate volume of DTT was added to the nucleic acid solution so that final concentration of DTT in the reaction solution is 50 mM. The reaction was carried out at room temperature for 1 hour. After 1 hour, gel filtration was performed using desalting spin column containing 7K molecular weight cut-off zeba resin buffer exchanged to 0.1M HEPES buffer (pH 7.4). An absorbance at 260 nanometer was measured and total amount of thiol-nucleic acid was calculated based on extinction coefficient and volume recovered.

Multi-functionalized maleimide-terminated PEG polymer with tripentaerythritol core purchased from Jenkem Technology (item #8ARM (TP)-MAL-20K; MW 20,000) was used as a carrier to synthesize poly-oligonucleotide conjugate. PEG polymer is dissolved in 0.1M HEPES buffer (pH 7.4). Five molar equivalent of thiol-nucleic acid was reacted with one molar equivalent of maleamide-terminated PEG carrier. The reaction was carried out at room temperature for 3 hours in the mixer and in 4° C. for 18 hours. The unreacted maleamide was quenched using 71.5 nanomoles of beta-mercaptoethanol in water for 15 minutes. Moreover, unreacted thiols were quenched using 0.1 millimoles of n-ethylmaleamide in water for 15 additional minutes. The reaction mixture was purified using a size-exclusion column with superdex200 resin (made by Cytiva) on a fast protein liquid chromatography (FPLC) system.

### Example 2

This example illustrates the detection of a target nucleic acid of low-risk human papillomavirus (HPV) using a probe composition includes a capture probe, a poly-oligonucleotide conjugate, and a label probe.

The capture probe is a mixture of multiple ssDNA probes which target the low-risk subtype 6 and 11 of human papillomavirus (HPV) as follows:

| | SEQ |
|---|---|
| | ID |
| SEQUENCE | NO |
| 5′-TTAGGGTAACATGTCTTCCATGCATGTTGT | 1 |
| AAGATGTGGTTGTCGTACTT-3′ | |
| 5′-CAGCAGTGTAGGCAGCGACCCTTCCACGTA | 2 |
| AAGATGTGGTTGTCGTACTT-3′ | |
| 5′-AATTTAGCTTTATGAACCGTGCCTTGGITA | 3 |
| AAGATGTGGTTGTCGTACTT-3′ | |
| 5′-AAATGTCTTGTTTAGTTTCTTCTTCAACAG | 4 |
| AAGATGTGGTTGTCGTACTT-3′ | |
| 5′-TTTCCATGAAATTCTAGGCAGCACGCGCAG | 5 |
| AAGATGTGGTTGTCGTACTT-3′ | |
| 5′-CTGCATATGGATAGCCGCCTCGAAACAGGA | 6 |
| AAGATGTGGTTGTCGTACTT-3′ | |
| 5′-AAACACACAATTAATTTGCAACGTATGCAT | 7 |
| AAGATGTGGTTGTCGTACTT-3′ | |
| 5′-GATAGATTAAACGTCTTGCACAACTGGTCT | 8 |
| AAGATGTGGTTGTCGTACTT-3′ | |
| 5′-TGGTCGTTGCAGACGTGGAGGCATTTGCAC | 9 |
| AAGATGTGGTTGTCGTACTT-3′ | |
| 5′-ATGTTTAGTGTTCCCAACAGAAGCTGTTGC | 10 |
| AAGATGTGGTTGTCGTACTT-3′ | |
| 5′-TTTAAAGGTTGTGAATCTTGTCCGTCCACT | 11 |
| AAGATGTGGTTGTCGTACTT-3′ | |
| 5′-TTAGGGTAACAAGTCTTCCATGCATGTTGT | 12 |
| AAGATGTGGTTGTCGTACTT-3′ | |
| 5′-CAGCAGTGTAAGCAACGACCCTTCCACTGG | 13 |
| AAGATGTGGTTGTCGTACTT-3′ | |
| 5′-TTTATGAAGCGTGCCTTTCCCAATATGTGC | 14 |
| AAGATGTGGTTGTCGTACTT-3′ | |
| 5′-ATCTTCATTAGTTTCTTCTTCTACTGTAGG | 15 |
| AAGATGTGGTTGTCGTACTT-3′ | |
| 5′-TTTCCCTTGCAGTTCTAAGCAACAGGCACA | 16 |
| AAGATGTGGTTGTCGTACTT-3′ | |
| 5′-GCTGCAAAGGGAAAGTTGTCTCGCCACACA | 17 |
| AAGATGTGGTTGTCGTACTT-3′ | |
| 5′-CCTTTAGGTTCTTATAGGCATATGCATATA | 18 |
| AAGATGTGGTTGTCGTACTT-3′ | |
| 5′-AAACACGCACTGAATTTGCAGAGTGTGCAA | 19 |
| AAGATGTGGTTGTCGTACTT-3′ | |
| 5′-GAAAGATTAAACGTCTTGCACAACTGGTCT | 20 |
| AAGATGTGGTTGTCGTACTT-3′ | |
| 5′-TGGATGTTGCAGACGTGGAGGCATCTTTAC | 21 |
| AAGATGTGGTTGTCGTACTT-3′ | |
| 5′-AAAGGTCTTGTAGTTGTCTGATGTCTCCGT | 22 |
| AAGATGTGGTTGTCGTACTT-3′ | |
| 5′-TGTGCACTCCACAACCAGTCGGACGTTGCT | 23 |
| AAGATGTGGTTGTCGTACTT-3′ | |
| 5′-AATGTTGTGTTAAAGGTTGTGAGTCTTGTT | 24 |
| AAGATGTGGTTGTCGTACTT-3′ | |
| 5′-CAGGGTCAGGAGGCTGCAGGTCTAGTACTA | 25 |
| AAGATGTGGTTGTCGTACTT-3′ | |

The sequence of the oligonucleotide of the poly-oligonucleotide conjugate is as follows (SEQ ID NO: 26):

5′-ACAACTTAACACAACTTAACAAAAAACAACTTAACACAACTTAACAA
AAAACAACTTAACACAACTTAACAAAAAACAACTTAACACAACTTAACAA
AAAACAACTTAACACAACTTAAC AAGTACGACAACCACATCTT -3′

The oligonucleotide contains 5 repeating sequences (underlined) which contribute to the signal amplification in final application of the conjugate. The 3′ end of the oligonucleotide contains a polynucleotide sequence (italic) complimentary to the 3′ end region (italic) of the capture probe, allowing the oligonucleotide to hybridize with the capture probe.

In this example, the poly-oligonucleotide conjugate was synthesized according to the procedure in Example 1. Herein, 5′-end of the oligonucleotide is thiol modified. To generate the poly-oligonucleotide conjugate, an appropriate polymer with enough maleimide groups was selected. The thiolated oligonucleotide reacted with maleimide containing polymer in a pre-determined molar ratio to produce poly-oligo conjugate. Synthesized conjugate was purified with size-exclusion chromatography and analyzed by high pressure liquid chromatography to ensure purity and conjugate-consistency.

The label probe contains digoxin at both ends and has the following sequence (SEQ ID NO: 27):

5′-GTTAAGTTGTGTTAAGTTGT-3′

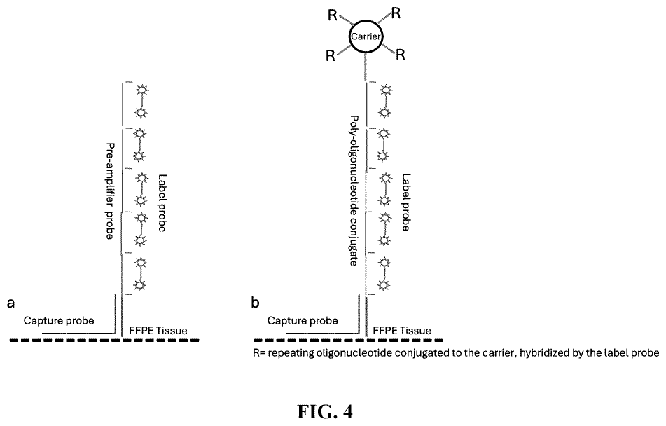

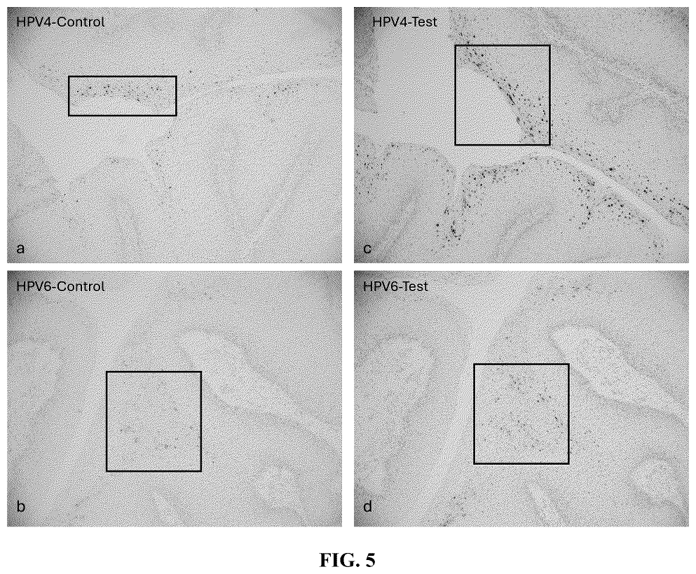

The probe composition was applied to an FFPE tissue sample that carries the low-risk subtypes 6 and 11 of the HPV virus along with a positive control as follows. The FFPE tissue slides underwent deparaffinization process followed by the antigen retrieval in Tris-EDTA buffer (PH=9.0) at 100° C. for 15 minutes. Capture probe was applied and allowed to hybridize at 37° C. for 2 hours. For control slides, a ssDNA pre-amplifier probe with the same sequence as the SEQ ID NO: 26 (2.5 nmol/L) is applied and hybridizes at 37° C. for 30 minutes (FIG. 4 a). For test slides, poly-oligonucleotide conjugate (2.5 nmol/L) was applied and allowed to hybridize for 30 minutes at 37° C. (FIG. 4 b). Further, both sets of slides were hybridized with the label probe (2.5 nmol/L) for 15 minutes at 37° C. After each hybridization step, slides were washed with wash buffer at room temperature. Next, the poly-horseradish peroxidase conjugated mouse-anti-digoxin antibody (component from Cat #CF6024-EBER Detection Kit, Celnovte Biotechnology, Inc.) was applied and incubated for 15 minutes at room temperature. Chromogenic detection was performed with 3,3′-diaminobenzidine (DAB) followed by counterstaining with hematoxylin (Celnovte Biotechnology, Inc.). Subsequently, dehydration and clearing steps were performed. The slides were then mounted and observed under a microscope. The test slides that hybridize with the poly-nucleotide conjugate demonstrated a higher degree of staining intensities compared to the control slides in all the tissues (FIG. 5). The overall staining result is summarized in the following table.

TABLE 1
Comparison of Poly-Oligonucleotide Conjugates and Positive Control
Group in Chromogenic In-situ Hybridization Experiment.

| | | Signal |
|---|---|---|
| Sample | Tissue ID | strength |
| A | HPV4 | + |
| | HPV6 | + |
| B | HPV4 | +++ |
| | HPV6 | +++ |

A: Control slides - hybridize with pre-amplifier probe after the capture probe step
B: Test slides - hybridize with poly-nucleotide conjugate after the capture probe step

For more detailed procedure of running the in-situ hybridization experiment with FFPE tissue samples, one can refer to the following literature: Wang, Fay, et al. “RNAscope: a novel in situ RNA analysis platform for formalin-fixed, paraffin-embedded tissues.” The Journal of molecular diagnostics 14.1 (2012): 22-29.

### Example 3

This example illustrates the detection of a target nucleic acid of low-risk human papillomavirus (HPV) using a probe composition includes a capture probe, a poly-oligonucleotide conjugate, an amplifier probe and a label probe. Herein, the label probe indirectly associated with the poly-oligonucleotide conjugate through the amplifier probe.

The sequences of the capture probe and the oligonucleotide of the poly-oligonucleotide conjugate are described in Example 2. The poly-oligonucleotide is synthesized as described in Example 2.

The sequence of the amplifier probe is as follows (SEQ ID NO: 28):

5′- ATACCTCGCTCTGCTAATCCAAAAAATACCTCGCTCTGCTAATCCA
AAAAATACCTCGCTCTGCTAATCCAAAAAATACCTCGCTCTGCTAATCCA
AAAAGTTAAGTTGTGTTAAGTTGT -3′

The label probe that contains digoxin at both ends and has the following sequence (SEQ ID NO: 29):

5′-GGATTAGCAGAGCGAGGTAT -3′

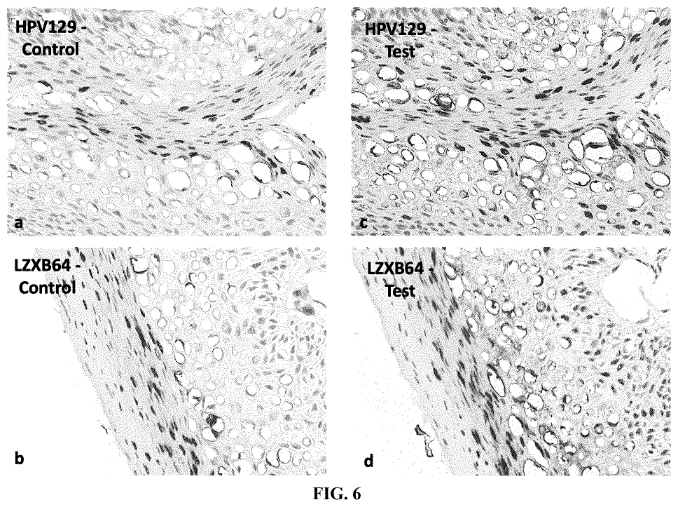

The probe composition is applied to an FFPE tissue sample that carries the low-risk subtypes 6 and 11 of the HPV virus along with a positive control as follows. The FFPE tissue slides undergo deparaffinization process followed by the antigen retrieval in Tris-EDTA buffer (PH=9.0) at 100° C. for 15 minutes. Capture probe is applied and allowed to hybridize at 37° C. for 2 hours. For control slides, a ssDNA pre-amplifier probe with the same sequence as SEQ ID NO: 26 in Example 2 (2.5 nmol/L) is applied and hybridizes at 37° C. for 30 minutes. For test slides, poly-oligonucleotide conjugate (2.5 nmol/L) is applied and allowed to hybridize for 30 minutes at 37° C. Both sets of slides are hybridized with the amplifier probe (2.5 nmol/L) for 30 minutes at 37° C. Further, both sets of slides are hybridized with label probe (2.5 nmol/L) for 15 minutes at 37° C. After each hybridization step, the slides are washed with wash buffer at room temperature. Next, the poly-horseradish peroxidase conjugated mouse-anti-digoxin antibody (component from Cat #CF6024-EBER Detection Kit, Celnovte Biotechnology, Inc.) is applied and incubated for 15 minutes at room temperature. Chromogenic detection is performed with 3,3′-diaminobenzidine (DAB) followed by counterstaining with hematoxylin (Celnovte Biotechnology, Inc.). Subsequently, dehydration and clearing steps are performed. The slides are then mounted and observed under a microscope. The test slides that hybridize with the poly-nucleotide conjugate demonstrated a higher degree of staining intensities compared to the control slides in all the tissues (FIG. 6). The overall staining result is summarized in Table 2.

TABLE 2
Comparison of Poly-Oligonucleotide Conjugates and Positive
Control Group in Chromogenic In-situ Hybridization Experiment,
where the label probe indirectly associated with the poly-
oligonucleotide conjugate through the amplifier probe.

| | | Signal |
|---|---|---|
| Sample | Tissue ID | strength |
| A | HPV129 | + |
| | LZXB64 | + |
| B | HPV129 | +++ |
| | LZXB64 | +++ |

A: Control slides - hybridize with the ssDNA pre-amplifier probe after the capture probe step
B: Test slides - hybridize with poly-nucleotide conjugate after the capture probe step

### Example 4

This example illustrates the detection of a biomarker, human Ki-67, using a nucleic acid conjugated antibody (mouse-anti-human Ki-67), a poly-oligonucleotide conjugate, and a label probe.

The sequence of the oligonucleotide to be conjugated to the antibody is as follows (SEQ ID NO: 30):

5′- AAAAAAAGATGTGGTTGTCGTACTT -3′

Herein, 5′-end of the oligonucleotide is amine modified. To generate oligo-antibody conjugates, the antibody is reduced by 20 mM 1,4-dithiothreitol (DTT) and purified by a 7K Zeba column (ThermoFisher Scientific). The oligonucleotide is first activated using 40 times molar equivalent of N-ε-malemidocaproyl-oxysuccinimide ester (EMCS) and purified by a 7K Zeba column. The resulting oligonucleotide and antibody products are mixed and incubated overnight before going through a 40K Zeba column for purification.

The sequence of the oligonucleotide of the poly-oligonucleotide conjugate is described in Example 2. The poly-oligonucleotide is synthesized the same way as mentioned by Example 2.

The label probe that contains fluorescence probe at both ends and has the following sequence (SEQ ID NO: 31):

5′-C12-GTTAAGTTGTGTTAAGTIGT-C7-3′

To generate fluorescence labelled oligonucleotides, 10 nmol of oligonucleotides (SEQ ID NO: 31) that has amine modification on both 5′ and 3′ ends is mixed with 40 nmol of Alexa Fluor 594 NHS (ThermoFisher Scientific) for 1 hour. The resulting product is purified by a 7K Zeba column.

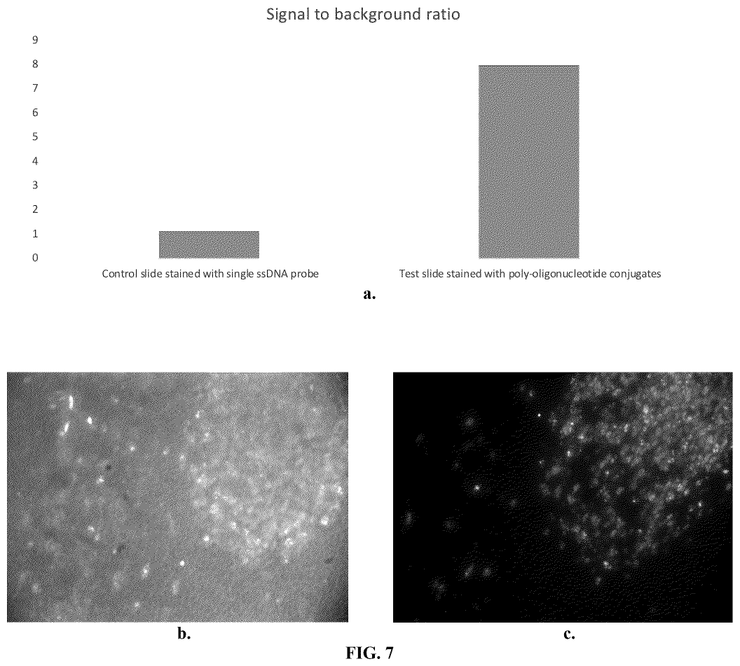

The oligo-antibody conjugate is applied to an FFPE tissue sample along with a positive control as follows. The FFPE tissue slides undergo deparaffinization process followed by the antigen retrieval in Tris-EDTA buffer (PH=9.0) at 100° C. for 15 minutes. For control and test samples, oligo-antibody conjugate is applied and incubated at room temperature (RT) for 1 hour. Tissue samples are then fixed for 30 mins using 2% PFA (paraformaldehyde) at RT. For control samples, a single ssDNA pre-amplifier probe (25 nmol/L) with the same sequence as SEQ ID NO: 26 in Example 2 is applied and hybridizes at 37° C. for 30 mins. For the test samples, a poly-oligonucleotide conjugate (25 nmol/L) is applied and allowed to hybridize for 30 minutes at 37° C. Next, control and test samples are hybridized with fluorescence labelled probe (50 nmol/L) for 1 hour at 37° C. After each hybridization step, the slides are washed with wash buffer at room temperature. After the dehydration step, the slides are then mounted and observed under a fluorescence microscope. The test slide and the control slide are then imaged under the same condition and compared (FIG. 7). The results indicate that the test slide demonstrate a much higher signal to background ratio than the control slide. The overall staining result is summarized in the following table. The median fluorescence intensity was calculated from segmented nucleus regions of the image and median background signal intensity was calculated from regions without cells. The signal to background ratio was calculated based on the ratio of the median fluorescence intensity from segmented nucleus over the median background signal intensity from regions without cells.

TABLE 3
Comparison of Poly-Oligonucleotide Conjugates and Positive
Control Group wherein the capture probe is a nucleic acid
conjugated antibody and the biomarker is a protein.

| | Median | | |
|---|---|---|---|
| | signal of | Meidian | Signal to |
| | positive | signal of | background |
| Sample | region | background | ratio |
| Control slide | 40 | 35 | 1.1 |
| stained with | | | |
| single ssDNA | | | |
| probe | | | |
| Test slide stained | 80 | 10 | 8.0 |
| with poly- | | | |
| oligonucleotide | | | |
| conjugates | | | |

1. A method of detecting a biomarker in a sample, said method comprising:
(a) applying a capture probe to the sample, wherein the capture probe specifically binds to the biomarker and comprises a nucleic acid having a tail polynucleotide sequence;
(b) applying a poly-oligonucleotide conjugate to the sample, wherein the poly-oligonucleotide conjugate comprises a carrier conjugated with a plurality of oligonucleotides, wherein each oligonucleotide comprises a first region comprising multiple repeating polynucleotide sequences, wherein at least one of the plurality of oligonucleotides comprises a second region capable of directly or indirectly associating with the tail polynucleotide sequence, thereby associating the poly-oligonucleotide conjugate with the capture probe bound to the target biomarker in step (a);
(c) applying a plurality of label probes to the sample, wherein each label probe capable of directly or indirectly associating with the repeating polynucleotide sequence, thereby associating the plurality of label probes with the poly-oligonucleotide conjugate bound to the capture probe in step (b); and
(d) detecting the label probes associated with the poly-oligonucleotide conjugate in step (c).

2. The method of claim 1, wherein the biomarker is a nucleic acid.

3. The method of claim 2, wherein the nucleic acid is selected from the group consisting of a genomic DNA, a cDNA, a mRNA, a rRNA, a miRNA, a Lnc RNA and a siRNA.

4. The method of claim 1, wherein the biomarker is a protein.

5. The method of claim 4, wherein the capture probe comprises an antibody against the protein.

6. The method of claim 1, wherein the tail polynucleotide sequence consists of 10-40 nucleotides.

7. The method of claim 1, wherein the carrier is a polymer, a nanoparticle, a bead, a liposome or a micelle.

8. The method of claim 7, wherein the polymer is selected from the group consisting of: polysaccharides such as dextrans and derivatized dextrans; vinyl polymers such as poly(acrylic acid), poly(acryl amides) and co-block polymers thereof; polyethylene glycol, polypropylene glycol and their derivatives; block co-polymers such as poly(ethylene oxide-co-propylene oxide); hyperbranched polymers such as PAMAM dendrimers; poly amino acids such as poly-lysine; proteins such as albumins and immunoglobulins; or a combination thereof.

9. The method of claim 1, wherein the repeating polynucleotide sequence consists of 10-40 nucleotides.

10. The method of claim 1, wherein the second region is capable of hybridizing to the tail polynucleotide sequence.

11. The method of claim 1, wherein the second region is capable of associating with the tail polynucleotide sequence via a bridge probe.

12. The method of claim 1, wherein the label probe has a sequence capable of hybridizing to the repeating polynucleotide sequence.

13. The method of claim 1, wherein the label probe is capable of associating with the repeating polynucleotide sequence via a bridge probe.

14. The method of claim 1, wherein the label probe comprises a detectable label.

15. The method of claim 14, wherein the detectable label is a fluorophore, a horse radish peroxidase or an alkaline phosphatase.

16. The method of claim 1, wherein the sample is a cell or tissue sample.

17-18. (canceled)


SEQUENCE LISTING (31 sequences)

SEQ ID NO: 1 - DNA, 50 bp, synthetic construct
  source 1..50: mol_type = other DNA
ttagggtaacatgtcttccatgcatgttgtaagatgtggttgtcgtactt

SEQ ID NO: 2 - DNA, 50 bp, synthetic construct
  source 1..50: mol_type = other DNA
cagcagtgtaggcagcgacccttccacgtaaagatgtggttgtcgtactt

SEQ ID NO: 3 - DNA, 50 bp, synthetic construct
  source 1..50: mol_type = other DNA
aatttagctttatgaaccgtgccttggttaaagatgtggttgtcgtactt

SEQ ID NO: 4 - DNA, 50 bp, synthetic construct
  source 1..50: mol_type = other DNA
aaatgtcttgtttagtttcttcttcaacagaagatgtggttgtcgtactt

SEQ ID NO: 5 - DNA, 50 bp, synthetic construct
  source 1..50: mol_type = other DNA
tttccatgaaattctaggcagcacgcgcagaagatgtggttgtcgtactt

SEQ ID NO: 6 - DNA, 50 bp, synthetic construct
  source 1..50: mol_type = other DNA
ctgcatatggatagccgcctcgaaacaggaaagatgtggttgtcgtactt

SEQ ID NO: 7 - DNA, 50 bp, synthetic construct
  source 1..50: mol_type = other DNA
aaacacacaattaatttgcaacgtatgcataagatgtggttgtcgtactt

SEQ ID NO: 8 - DNA, 50 bp, synthetic construct
  source 1..50: mol_type = other DNA
gatagattaaacgtcttgcacaactggtctaagatgtggttgtcgtactt

SEQ ID NO: 9 - DNA, 50 bp, synthetic construct
  source 1..50: mol_type = other DNA
tggtcgttgcagacgtggaggcatttgcacaagatgtggttgtcgtactt

SEQ ID NO: 10 - DNA, 50 bp, synthetic construct
  source 1..50: mol_type = other DNA
atgtttagtgttcccaacagaagctgttgcaagatgtggttgtcgtactt

SEQ ID NO: 11 - DNA, 50 bp, synthetic construct
  source 1..50: mol_type = other DNA
tttaaaggttgtgaatcttgtccgtccactaagatgtggttgtcgtactt

SEQ ID NO: 12 - DNA, 50 bp, synthetic construct
  source 1..50: mol_type = other DNA
ttagggtaacaagtcttccatgcatgttgtaagatgtggttgtcgtactt

SEQ ID NO: 13 - DNA, 50 bp, synthetic construct
  source 1..50: mol_type = other DNA
cagcagtgtaagcaacgacccttccactggaagatgtggttgtcgtactt

SEQ ID NO: 14 - DNA, 50 bp, synthetic construct
  source 1..50: mol_type = other DNA
tttatgaagcgtgcctttcccaatatgtgcaagatgtggttgtcgtactt

SEQ ID NO: 15 - DNA, 50 bp, synthetic construct
  source 1..50: mol_type = other DNA
atcttcattagtttcttcttctactgtaggaagatgtggttgtcgtactt

SEQ ID NO: 16 - DNA, 50 bp, synthetic construct
  source 1..50: mol_type = other DNA
tttcccttgcagttctaagcaacaggcacaaagatgtggttgtcgtactt

SEQ ID NO: 17 - DNA, 50 bp, synthetic construct
  source 1..50: mol_type = other DNA
gctgcaaagggaaagttgtctcgccacacaaagatgtggttgtcgtactt

SEQ ID NO: 18 - DNA, 50 bp, synthetic construct
  source 1..50: mol_type = other DNA
cctttaggttcttataggcatatgcatataaagatgtggttgtcgtactt

SEQ ID NO: 19 - DNA, 50 bp, synthetic construct
  source 1..50: mol_type = other DNA
aaacacgcactgaatttgcagagtgtgcaaaagatgtggttgtcgtactt

SEQ ID NO: 20 - DNA, 50 bp, synthetic construct
  source 1..50: mol_type = other DNA
gaaagattaaacgtcttgcacaactggtctaagatgtggttgtcgtactt

SEQ ID NO: 21 - DNA, 50 bp, synthetic construct
  source 1..50: mol_type = other DNA
tggatgttgcagacgtggaggcatctttacaagatgtggttgtcgtactt

SEQ ID NO: 22 - DNA, 50 bp, synthetic construct
  source 1..50: mol_type = other DNA
aaaggtcttgtagttgtctgatgtctccgtaagatgtggttgtcgtactt

SEQ ID NO: 23 - DNA, 50 bp, synthetic construct
  source 1..50: mol_type = other DNA
tgtgcactccacaaccagtcggacgttgctaagatgtggttgtcgtactt

SEQ ID NO: 24 - DNA, 50 bp, synthetic construct
  source 1..50: mol_type = other DNA
aatgttgtgttaaaggttgtgagtcttgttaagatgtggttgtcgtactt

SEQ ID NO: 25 - DNA, 50 bp, synthetic construct
  source 1..50: mol_type = other DNA
cagggtcaggaggctgcaggtctagtactaaagatgtggttgtcgtactt

SEQ ID NO: 26 - DNA, 140 bp, synthetic construct
  source 1..140: mol_type = other DNA
acaacttaacacaacttaacaaaaaacaacttaacacaacttaacaaaaaacaacttaacacaacttaacaaaaaacaacttaacacaacttaacaaaaaacaacttaacacaacttaacaagtacgacaaccacatctt

SEQ ID NO: 27 - DNA, 20 bp, synthetic construct
  source 1..20: mol_type = other DNA
gttaagttgtgttaagttgt

SEQ ID NO: 28 - DNA, 120 bp, synthetic construct
  source 1..120: mol_type = other DNA
atacctcgctctgctaatccaaaaaatacctcgctctgctaatccaaaaaatacctcgctctgctaatccaaaaaatacctcgctctgctaatccaaaaagttaagttgtgttaagttgt

SEQ ID NO: 29 - DNA, 20 bp, synthetic construct
  source 1..20: mol_type = other DNA
ggattagcagagcgaggtat

SEQ ID NO: 30 - DNA, 25 bp, synthetic construct
  source 1..25: mol_type = other DNA
aaaaaaagatgtggttgtcgtactt

SEQ ID NO: 31 - DNA, 22 bp, synthetic construct
  source 1..22: mol_type = other DNA
cgttaagttgtgttaagttgtc